(19) United States
(12) Patent Application Publication
(10) Pub. No.: US 2010/0104533 A1
(43) Pub. Date: Apr. 29, 2010

(54) EARLY INTERVENTION OF VIRAL INFECTION WITH IMMUNE ACTIVATORS

(76) Inventors: William A. Carter, Spring City, PA (US); David Strayer, Bryn Mawr, PA (US)

Correspondence Address: NIXON & VANDERHYE, PC, 901 NORTH GLEBE ROAD, 11TH FLOOR, ARLINGTON, VA, 22203, US

(21) Appl. No.: 12/452,179

(22) PCT Filed: Jun. 18, 2008
(86) PCT No.: PCT/US08/07529

§ 371 Date: Dec. 18, 2009

Related U.S. Application Data
(60) Provisional application No. 60/929,203, filed on Jun. 18, 2007.

(51) Int. Cl. A61K 38/21; A61K 31/7105; A61P 31/12; A61P 37/04

(52) U.S. Cl. 424/85.7; 514/44 R

(57) ABSTRACT

Symptoms of viral infection are mitigated by administering to a subject exposed to a virus a protective or symptom-mitigating amount of a dsRNA and continuing administration until the subject's symptoms have improved.

### CROSS-REFERENCE TO RELATED APPLICATIONS

This application claims the priority benefit of provisional U.S. Application No. 60/929,203, filed Jun. 18, 2007.

### FIELD OF THE INVENTION

The invention relates to the use of immune activators deployed either prior to (i.e., prophylactically) or at a time when the initial signs of viral infection are observed in a specific population of subjects, and before the viral load in the patients increases, in order to obtain a disproportionately strong effect on increasing and/or maintaining survival by protecting the blood brain barrier via a non-toxic set of immune actions. The result effectiveness is preserved against multiple virulent strains of otherwise lethal viruses.

### BACKGROUND OF THE INVENTION

In the past, intranasally administered therapeutic agents in the treatment of viral infections have not been employed. A well documented study was conducted using an inactivated intranasal influenza vaccine. At the time), many investigators considered the nasal route to be the most effective route for inducing both mucosal and systemic immunity to an infectious agent. And when the inactivated influenza vaccine was administered intranasally, clinically significant antibody responses to influenza virus were elicited by the vaccine but a significant number of recipients developed Bell's palsy. The product, which was authorized for use in Switzerland, was then promptly withdrawn from the market. See Mutsch et al. (N. Engl. J. Med. 350(9):896-903, 2004) and the editor's comments and perspective at pages 860-861. Because of these events and other considerations (including intranasal bleeding and severe local irritation), the intranasal route of administration for antiviral substances has largely been avoided.

Contrary to Mutsch's experience, we have found that another therapeutic agent dsRNA (e.g., AMPLIGEN®) may be safely administered preferably intranasally or otherwise to patients on a prophylactic basis or at the first signs of symptomology in a defined population of subjects without concern for toxicity and is effective therapy against a viral infection, in particular when therapy is commenced at an early stage in the viral infection. Early signs of a viral infection may include low grade fever and easy fatiguability.

### SUMMARY OF THE INVENTION

It is an object of the invention to provide one or more immune activators (e.g., dsRNA and/or interferon) to a subject (e.g., a human or other animal) who is in need of treatment (i.e., exposed to a virus or at risk of such exposure).

The immune activator(s) may be administered to a subject in need of treatment immediately prior to exposure to the virus, when the initial signs of viral infection are observed, or at any time(s) thereabout (e.g., administering one or more doses within a range of time from 48 hours before to 48 hours after exposure, preferably within 24 hours of exposure, prior to 24 hours after exposure, or prior to 12 hours after exposure). Alternatively, the one or more immune activators may be administered before the viral load in the subject increases to cause severe symptoms of viral infection (i.e., severe symptomatology). The immune activator(s) are preferably well tolerated when applied intranasally to the subject. They may be co-administered locally (e.g., intranasally or another oral-mucosal route) at the same time or sequentially.

The viral titer may be reduced by at least 90% in the subject's blood, a tissue which is the target for infection by the virus, or any other conveniently assayed sample (e.g., nasal wash for a virus affecting the respiratory system) as compared to a subject who was not treated.

In one aspect of the invention, the symptoms of a viral infection are mitigated by administering to a subject exposed to an amount of one or more dsRNAs effective to protect against viral infection or to mitigate the symptoms associated therewith. The administration of dsRNAs may be continued for at least from 24 hours to 72 hours, or until the subject's symptoms have improved.

dsRNAs may be administered to enhance the integrity of the subject's blood-brain barrier, thereby thwarting further internal migration of viruses entering the body via nasal pathways and oral compartments of the subject's body. The dsRNA may act specifically via TLR3 receptors within the subject's airways to effectively incapacitate multiple genetic variants of otherwise lethal viruses. dsRNAs may prevent migration of vaccine components into olfactory bulb and other cranial nerves of the subject.

In another aspect, a medicament (e.g., pharmaceutical composition) containing the immune activator(s) is provided. Optional other components of the medicament include excipients and a vehicle (e.g., aqueous buffer or water for injection) packaged aseptically in one or more separate containers (e.g., nasal applicator or injection vial). Processes for using and making the medicament are also provided. Further aspects will be apparent from the following description and claims, and any generalizations thereto.

DESCRIPTION OF SPECIFIC EMBODIMENTS OF THE INVENTION

Although immune activators are routinely used in the treatment or prevention of viral infections, they are not able to pass the blood brain barrier (BBB). The dsRNAs, however, particularly AMPLIGEN®, may be conveniently supplied to the brain without difficulties. It has been postulated that various disease-causing viruses are in part responsible for significant mortality as they are able to enter the brain through the BBB. Thus procedures to enhance the BBB's resistance to viral infiltration and limit the BBB's permeability and related possible inflammation of the involved tissue are thought to enhance the successful outcome of subsequent antiviral therapy.

In one study changes in the permeability of the BBB were evaluated in two mouse models of viral encephalitis. The ability of sodium fluorescein (NaFl) to cross the BBB from the serum into the central nervous system was assayed in animals inoculated with virulent strains of either Banzi or Semliki Forest viruses. To test the hypothesis that increases in BBB permeability were associated with poor disease outcome, subsequent experiments measured BBB permeability in conjunction with treatment with the interferon inducer AMPLIGEN® (poly I:poly C₁₂U). A single intraperitoneal injection of AMPLIGEN® (1 mg/kg) administered either 24 hrs or 4-6 hrs before, but not 24 hrs after, virus inoculation with Banzi virus provided significant improvements in survival, viral brain titers, weight change and BBB permeability. In comparison, a similar treatment with AMPLIGEN® administered either 24 hrs or 4-6 hrs before inoculation with Semliki Forest virus was able to significantly improve weight change and BBB permeability, but only animals receiving AMPLIGEN® 4-6 hrs pre-virus inoculation showed a significantly improved mortality. In general, it was found that evaluation of BBB permeability was a more sensitive indicator of disease outcome and the antiviral efficacy of AMPLIGEN® than either weight change or brain virus titers.

There is a rationale for our early drug intervention. Studies of viral pathogenesis have clearly demonstrated that the first step in pathogenesis is entry of virus into the host subject. One of the main routes of entry in humans is via the respiratory tract. The respiratory tract is populated with epithelial cells and dendritic cells. Epithelial cells posses a variety of molecular surface structures, which may serve as cell receptors that interact with viral attachment proteins. Dendritic cells which act as “sentinel cells” posses molecular surface structures that recognize pathogen-associated molecular patterns (PAMPs). These PAMPs include a set of Toll-like receptors (TLRs) that specifically recognize double-stranded RNAs. This TLR is known as Toll-like receptor 3 (TLR3).

As known, influenza virus may cause disease in the upper and lower respiratory tracts. Natural infection by this virus is usually initiated with relatively small inoculums of virion (e.g., 10²-10⁵ PFU). Upon entry into the respiratory tract via the oral cavity or the nasal passage, virions of influenza virus will be dispersed among susceptible epithelial cells and dendritic cells. This is a series of stochastic events that result in the initial successful infection of relatively few cells. Administration of ALFERON LDO® or AMPLIGEN® by the oral-mucosal route during this very early phase of viral pathogenesis would significantly influence the outcome of infection by direct or indirect inhabitation of viral multiplication in infected cells. Non-infected epithelial and dendritic cells can be induced to produce α/β-IFNs through the action of ALFERON LDO® and/or AMPLIGEN® (which is a powerful inducer of the antiviral state via several different mechanisms).

The key point to recognize in this strategy is that the development of clinical disease as an outcome of naturally occurring infection requires amplification of the initial viral inoculum within the host. Clearly, early administration of ALFERON LDO® and/or AMPLIGEN® effectively reduces the number of susceptible cells that will be infected in later rounds of infection.

Since avian A/H5 N1 influenza virus is known to infect cells in the gastric mucosa, it is very important to intervene with effective drugs that can act locally in the respiratory tract and systemically. Amplification of virus can occur in a number of organs and tissues. Usually primary or secondary viremias occur between rounds of viral multiplication. Early and sustained therapeutic intervention is expected to reduce the severity and duration of disease.

We have found that low levels of virus cause viral infections and, as the virus replicates in other tissues, the viral load builds. During this time symptoms of viral infection progressively occur. The level of virus in the subject receiving therapy is critically important as a high viral load may overcome the antiviral effect(s) of a therapeutic agent. In certain situations, low levels of virus are encountered and, when symptoms appear, are treated with an antiviral drug. In accordance with the invention, a drug that may become ineffective against a higher viral load will show efficacy at a lower viral load and thus be effective.

The mismatched dsRNA may be of the general formula rIₙ·r(C₁₁₋₁₄U)ₙ, which is preferably rIₙ·r(C₂U)ₙ, where n is an integer having a value of from 40 to 40,000. In this and the other formulae that follow r=ribo. Other mismatched dsRNAs for use in the present invention are based on co-polynucleotides selected from poly (Cₘ,U) and poly (Cₘ,G) in which m is an integer having a value of from 4 to 29 and are mismatched analogs of complexes of polyriboinosinic and polyribocytidilic acids, formed by modifying rIₙ·rCₙ to incorporate unpaired bases (uracil or guanine) along the polyribocytidylate (rCₘ) strand. Alternatively, the dsRNA may be derived from r(I)·r(C) dsRNA by modifying the ribosyl backbone of polyriboinosinic acid WO, e.g., by including 2′-O-methyl ribosyl residues. The mismatched may be complexed with an RNA-stabilizing polymer such as lysine cellulose. Of these mismatched analogs of rIₙ·rCₙ, the preferred ones are of the general formula rIₙ·r(C₁₁₋₁₄,U)ₙ or rIₙ·r(C₂₉,G)ₙ, and are described by Ts'o & Carter in U.S. Pat. Nos. 4,024,222 and 4,130,641; the disclosures of which are hereby incorporated by reference. The dsRNAs described therein are generally suitable for use according to the present invention.

Other examples of mismatched dsRNA for use in the invention include:

r(I)·r(C₄, U),

r(I)·r(C₇, U),

r(I)·r(C₁₃, U),

r(I)·r(C₂₂, U),

r(I)·r(C₂₀, G) and

r(I)·r(C_{p·23},G_{>p}).

For a subject (e.g., 150 lb human) the dose of dsRNAs prior to infection or the onset of symptoms of viral infection may range from 0.1 to 25,000 μg, preferably from 0.5 to 5,000 μg. Once symptoms of viral infection in the subject develop, the dose of dsRNAs may range from 0.1 to 25,000 μg, preferably from 0.5 to 5,000 μg. Intranasal administration is preferred.

Alternatively the dsRNA may be matched (i.e., not in mismatched form). Thus polyadenylic acid complexed with polyuridylic acid (poly A·poly U) may also be used. The matched dsRNAs may be administered intravenously, intramuscularly, intranasally or topically.

Formulations for administration include aqueous solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavoring, coloring and/or sweetening agents. They may be applied nasally with a spray or nebulizer. It will be appreciated that the preferred route will vary with the condition and age of the recipient, the nature of the infection and the chosen active ingredient.

The optional α-interferon component of the invention is preferably ALFERON N® Injection the only approved natural, multi-species, α-interferon available in the United States. It is the first natural source, multi-species interferon and is a consistent mixture of at least seven species of α-interferon. The interferon is preferably a natural cocktail of at least seven species of human α-interferon. In contrast, the other available α-interferons are single molecular species of α-interferon made in bacteria using DNA recombinant technology. These single molecular species of α-interferon also lack an important structural carbohydrate component because this glycosylation step is not performed during the bacterial process.

Unlike species of α-interferon produced by recombinant techniques, ALFERON N® Injection is produced by human white blood cells which are able to glycosylate the multiple α-interferon species. Reverse phase HPLC studies show that ALFERON N® Injection is a consistent mixture of at least seven species of alpha interferon (α2, α4, α7, α8, α10, α16 and α17). This natural-source interferon has unique anti-viral properties distinguishing it from genetically engineered interferons. The high purity of ALFERON N® Injection and its advantage as a natural mixture of seven interferon species, some of which, like species 8b, have greater antiviral activities than other species, for example, species 2b, which is the only component of INTRON A®. The superior antiviral activities, for example in the treatment of chronic hepatitis C virus (HCV) and HIV infection, and tolerability of ALFERON N® Injection compared to other available recombinant interferons, such as INTRON A® and ROFERON A®, have been reported. ALFERON N® Injection is available as an injectable solution containing 5,000,000 international units (IU) per ml. For internal administration the α-interferon may, for example, be formulated in conventional manner for oral, nasal or buccal administration. Formulations for oral administration include aqueous solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavoring, coloring and/or sweetening agents. α-Interferon may be administered preferably by a suitable route including oral, nasal, parenteral (including injection) or topical (including transdermal, buccal and sublingual). It will be appreciated that the preferred route will vary with the condition and age of the recipient, the nature of the infection and the chosen active ingredient.

The recommended dosage of the components will depend on the clinical status of the patient and the experience of the clinician in treating similar infection. As a general guideline, dosage of ALFERON N® Injection utilized for systemic infections is 5 to 10 million units (e.g., subcutaneous injection) three times weekly. Experience to date is with dosages above 3 IU/lb of patient body weight. Oral α-interferon (ALFERON LDO®) has been administered as a liquid solution in the range of 500-10,000 IU/day and calculated on the basis of a 150 pound human this is from 3.3 to 66.0 IU/lb per day.

Our experience indicates beneficial results are obtained at dosage levels of α-interferon in excess of 450 IU, that is greater than 3 IU/pound body weight. These amounts are in contrast to and greater than Cummin's use in U.S. Pat. No. 5,910,304 of alpha interferon administration to the pharyngeal mucosa orally or as lozenge or tablet.

Exposure of the subject's oromucosa to low doses of α-interferon is reported to lead to biological effects in humans and animals. A healthcare provider would be able, however, to determine the optimal dose and schedule of low dose oral α-interferon to achieve a systemic antiviral effect. A naturally derived alpha α-interferon (ALFERON N® Injection) has been approved for treatment of Condylomata acuminata. It is active at doses significantly lower than those used for recombinant α-interferon.

### EXAMPLE 1

The potential toxicity of AMPLIGEN® was evaluated after a 4-week repeated intranasal instillation in male and female CD-1 mice. Results showed it did not give the toxicity previously reported with other TLR activators.

Three groups of mice were treated with 0.4, 2 and 10 μg/injection dose levels once weekly for four consecutive weeks. A control group was instilled with 0.9% sodium chloride. Mice were observed for morbidity and mortality, clinical signs and changes in body weight during the in-life phase of the study. Measurements of clinical pathology parameters, organ weight, gross necropsy finding and histopathology were assessed post mortem.

No AMPLIGEN®-related findings were observed in morbidity and mortality, clinical signs, body weight, organ weights, and clinical pathology parameters.

Treatment related effects were limited to minimal alveolar macrophage hyperplasia that was seen in all treatment groups, including the control group. However, alveolar macrophage hyperplasia is a typical response of the lung to instilled particles in the lung. Although the incidence was more prevalent in the higher dose groups, the trend was not statistically significant.

Intranasal administration of Ampligen® at concentrations up to 10 μg/instillation had no effect on male and female CD-1 mice after four weeks of repeated dose treatment.

### EXAMPLE 2

The activity of AMPLIGEN® against influenza A virus in mice was studied using two strains of duck virus H5N1 and two strains of influenza virus H1N1. Mice were challenged with various levels of virus. AMPLIGEN® was administered prior to administration of the influenza virus and the results are shown in the following Table.

Activity of dsRNA Against Influenza A Virus Infection In Mice

| | | Dose | | | |
|---|---|---|---|---|---|
| Influenza | Dosage | Level | % Survival | | |
| Strain | Route | (mg/kg) | Control | dsRNA | Difference |
| A/Duck/MN/1525/81 | IN | 1 | 0 | 40 | 40 |
| H5N1 | | | | | |
| A/Duck/MN/1525/81 | IP | 20 | 15 | 40 | 25 |
| H5N1 | | | | | |
| A/PR/8/34 H1N1 | IN | 3 | 0 | 60 | 60 |
| A/PR/8/34 H1N1 | IN | 3 | 37.5 | 75 | 37.5 |

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A method of mitigating the symptoms of a viral infection comprising administering to a subject exposed to a virus a protective or symptom-mitigating amount of a dsRNA and continuing administration until the subject's symptoms have improved.

2. The method according to claim 1, wherein the dsRNA is administered before the subject's viral load reaches levels as assessed by substantial symptomatology.

3. The method according to claim 1, wherein the dsRNA is mismatched.

4. The method according to claim 3, wherein the dsRNA is rIₙ·r(C₁₁₋₁₄U)ₙ, Poly A·Poly U or rIₙ·r(C₂₉,G)ₙ, in which n is an integer having a value of from 40 to 40,000 and r is ribo.

5. The method according to claim 1, wherein the dsRNA is administered nasally or by another oral-mucosal route to the subject.

6. The method according to claim 5, wherein the dsRNA is administered to enhance the integrity of the subject's blood-brain barrier, thereby thwarting further internal migration of viruses entering the subject's body via nasal pathways and oral compartments of the subject's body.

7. The method according to claim 1, wherein the dsRNA, acting specifically via TLR3 receptors within the subject's airways effectively incapacitates multiple genetic variants of otherwise lethal viruses.

8. The method according to claim 1, wherein the dsRNA is well tolerated applied intranasally to the subject.

9. The method according to claim 1, wherein the dsRNA prevents migration of vaccine components into olfactory bulb and other cranial nerves of the subject.

10. The method according to claim 1 further comprising co-administering locally the dsRNA with a natural cocktail of human alpha interferons.

11-12. (canceled)

13. A method of treatment comprising administering to a subject exposed to a virus exposed to a virus before or after said treatment a protective- or symptom-mitigating amount of a dsRNA and continuing administration until the subject's symptoms have improved.

14. The method according to claim 13, wherein the dsRNA is administered before the subject's viral load reaches levels as assessed by substantial symptomatology.

15. The method according to claim 13, wherein the dsRNA is mismatched.

16. The method according to claim 15, wherein the dsRNA is rIₙ·r(C₁₁₋₁₄U)ₙ, Poly A·Poly U or rIₙ·r(C₂₉,G)ₙ, in which n is an integer having a value of from 40 to 40,000 and r is ribo.

17. The method according to claim 13, wherein the dsRNA is administered nasally or by another oral-mucosal route to the subject.

18. The method according to claim 5, wherein the dsRNA is administered to enhance the integrity of the subject's blood-brain barrier, thereby thwarting further internal migration of viruses entering the subject's body via nasal pathways and oral compartments of the subject's body.

19. The method according to claim 13, wherein the dsRNA, acting specifically via TLR3 receptors within the subject's airways effectively incapacitates multiple genetic variants of otherwise lethal viruses.

20. The method according to claim 13, wherein the dsRNA is well tolerated applied intranasally to the subject.

21. The method according to claim 13, wherein the dsRNA prevents migration of vaccine components into olfactory bulb and other cranial nerves of the subject.

22. The method according to claim 13 further comprising co-administering locally the dsRNA with a natural cocktail of human alpha interferons.
